(19) United States
(12) Patent Application Publication
(10) Pub. No.: US 2007/0249891 A1
(43) Pub. Date: Oct. 25, 2007

(54) Ceiling newsletter system and method

(76) Inventor: Thomas Moore Davis, Mt. Pleasant, SC (US)

Correspondence Address: KATHLEEN M HARLESTON;THE HARLESTON LAW FIRM, 909 TALL PINE ROAD, MT PLEASANT, SC, 29464, US

(21) Appl. No.: 11/409,160

(22) Filed: Apr. 21, 2006

(51) Int. Cl. A61M 21/00

(52) U.S. Cl. 600/27; 600/26

(57) ABSTRACT

A method for easing stress on a patient in a stressful treatment area, including the steps of: (a) providing at least one page of a periodically issued ceiling newsletter; (b) mounting at least two members of an attachment mechanism on a ceiling of the treatment area; (c) mounting at least two corresponding members of the attachment mechanism on a back side of each ceiling newsletter page; and (d) removably mounting the ceiling newsletter on the treatment area ceiling. Also included herein is a method including the steps of: a) subscribing to a ceiling newsletter; b) transcribing subscriber information to a customer list; c) mailing the ceiling newsletter issues to the subscribers periodically; and d) receiving the issue, affixing corresponding attachment mechanism members to the ceiling and to the back of each page of the ceiling newsletter, and removably attaching the ceiling newsletter to the treatment area ceiling.

### BACKGROUND OF THE INVENTION

1. Technical Field

The present invention relates to a method and system including a ceiling newsletter for reducing stress in a stressful environment, such as a physician's examination room, a health care clinic, a dentist's office, or the like.

2. Background Information

Oftentimes, visiting a doctor, dentist, or the like can entail a wait of several hours. This time can be as stressful as the actual treatment or examination period, particularly when one has been admitted to the sterile examination room and is awaiting the arrival in the examination room of the health care provider. Once the patient has examined the wall chart showing a skeleton, inner ear, or other part of the body, and perhaps toyed with the tongue depressors, there is not much else to do except to imagine the worst. The patient's discomfort is only increased when he or she is partially clad in a drafty examination gown. Where the health care provider is a dentist or gynecologist, for example, the patient's discomfort often continues after the doctor, dentist, or other health care provider arrives as the patient undergoes the examination or treatment. The dentist, for example, often performs an initial procedure, such as a shot of anesthetic, and then leaves the treatment room to attend to other patients in other examining rooms.

The present invention centers around an amusing, colorful, easy to read ceiling newsletter that helps to keep the patient in a good frame of mind and encourages the patient to continue to lie still on the examining table or in the chair during treatment. The patient is more likely to be thinking about the reading material or visual display in the ceiling newsletter than dreading the doctor's examination, listening to the dentist's drill, etc. This distraction is of benefit to the health care practitioner and the health care assistant as well as the patient, since it enhances the patient's comfort level and permits the examination or treatment to go more smoothly.

### BRIEF SUMMARY OF THE INVENTION

The present invention is a stress reduction business method and system. The method comprises the steps of:
(a) providing at least one page of a ceiling newsletter;
(b) mounting at least one member of an attachment mechanism on a back side of the at least one page ceiling newsletter; and
(c) removably mounting the at least one page of the ceiling newsletter on a ceiling above the patient treatment area, with the at least one attachment mechanism member corresponding to a corresponding attachment mechanism member mounted on the ceiling.

The present invention also includes a method for distributing a ceiling newsletter, comprising the steps of:
a) Subscribing to a ceiling newsletter;
b) Transcribing subscriber information from the new subscriber to a customer list;
c) Mailing an issue of the ceiling newsletter and corresponding attachment mechanism members to each subscriber periodically; and
d) Receiving the issue of the ceiling newsletter, affixing corresponding attachment mechanism members to the ceiling of a treatment area and to the back of each page of the ceiling newsletter, and removably attaching the issue of the ceiling newsletter to the treatment area ceiling.

Also included herein is a newsletter system for easing stress on a patient in a high stress treatment area, comprising:
(a) at least one page of a periodically issued ceiling newsletter;
(b) at least two sets of hook and loop strips attachable to a back of the at least one page of the ceiling newsletter;
(c) at least two sets of corresponding hook and loop strips attachable to a ceiling over the treatment area; and
(d) at least one miniature of the ceiling newsletter.

### BRIEF DESCRIPTION OF THE SEVERAL VIEWS OF THE DRAWINGS

A more complete understanding of the invention and its advantages will be apparent from the following detailed description taken in conjunction with the accompanying drawings, wherein examples of the invention are shown, and wherein:

FIG. 1 is a perspective view of a dentist's treatment room setting according to the present invention, showing a ceiling newsletter on the ceiling of the room;

FIG. 2 is a perspective view of an obstetrician/gynecologist's examination room setting according to the present invention, showing a ceiling newsletter on the ceiling;

FIG. 3 is a perspective view of a blood donation center setting according to the present invention, showing a ceiling newsletter on the ceiling;

FIG. 4 is an illustrative example of a ceiling newsletter layout generated in accordance with the present invention, shown without copy;

FIG. 5 is a basic block diagram of a ceiling newsletter method according to the present invention;

FIG. 6 is a basic block diagram of a ceiling newsletter method according to the present invention;

FIG. 7 is a perspective view of a ceiling newsletter according to the present invention, shown with hook and loop strips;

FIG. 8 is a side elevational view of a ceiling newsletter on a ceiling according to the present invention;

FIG. 9 is an illustrative example of a first page of a ceiling newsletter generated in accordance with the present invention; and

FIG. 10 is an illustrative example of a second page of the ceiling newsletter of FIG. 9.

### DETAILED DESCRIPTION OF THE INVENTION

In the following description, like reference characters designate like or corresponding parts throughout the several views. Also, in the following description, it is to be understood that such terms as “front,” “back,” “within,” and the like are words of convenience and are not to be construed as limiting terms. Referring in more detail to the drawings, the invention will now be described.

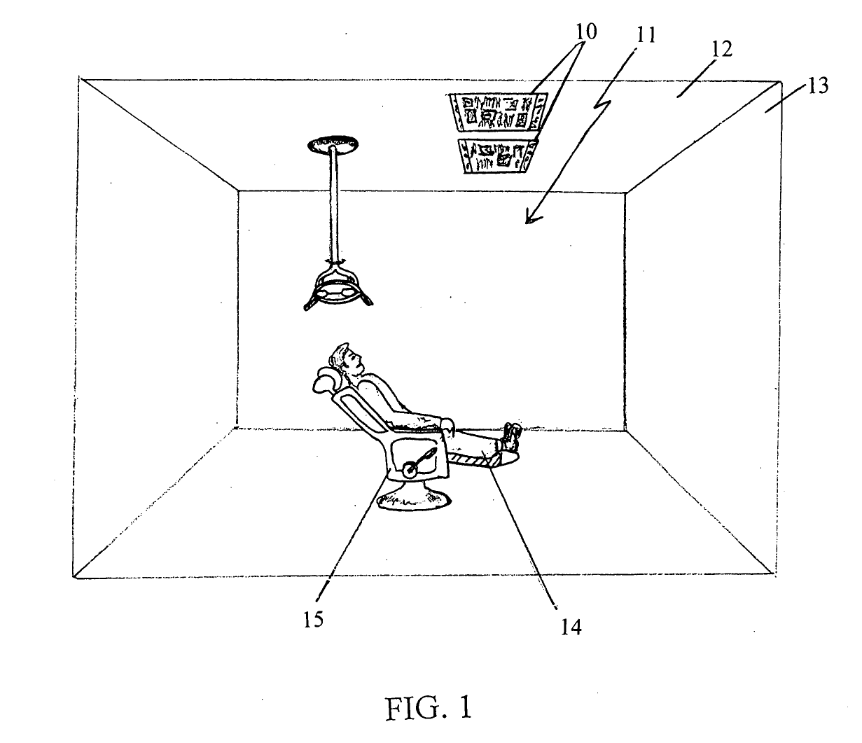

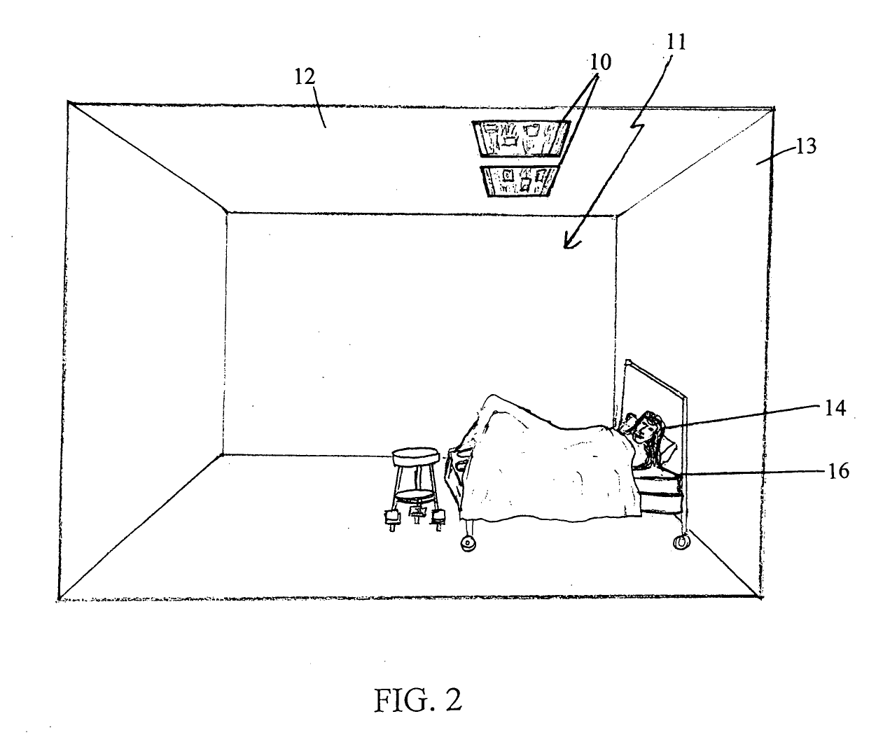

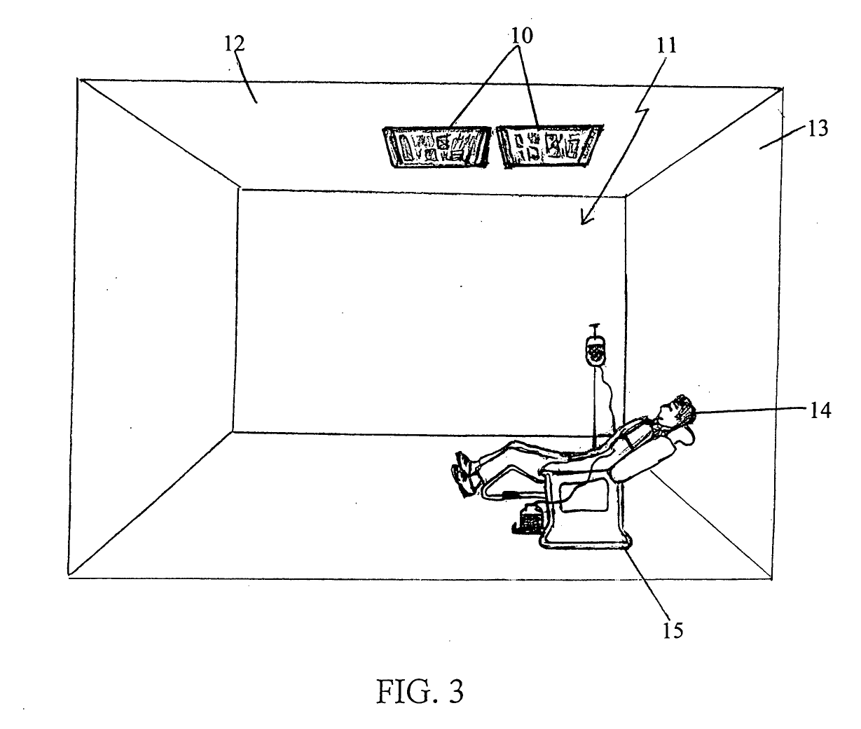

Turning first to FIGS. 1 through 3, a dentist's treatment room setting (see FIG. 1), an obstetrician/gynecologist's examination room setting (see FIG. 2), and a blood donation center setting (see FIG. 3) according to the present invention show an issue of a ceiling newsletter 10 mounted on the ceiling 12 of a treatment room 13 over the treatment area 11 for a patient 14 to view prior to and during the respective treatment. The ceiling newsletter 10 is a monthly newsletter that is attachable to a ceiling 12 of an examination or treatment room 13, or any other room where a stressful examination or treatment may take place. Its purpose is to act as a distraction and entertainment for those who are, for various reasons, obliged to look up at a ceiling for a protracted period of time, as would a patient in a dentist's chair, an obstetrician/gynecologist's examination room, an optometrist's office/vision center, blood donation center, etc. Although it is intended for patient who have to lie down for treatment, or who are otherwise obliged to look up at a ceiling, it can also be suitable for use in a waiting room in a physician's or dentist's office, veterinarian's office, or anywhere people are relatively confined to a chair in a waiting room.

By “treatment area” 11 is meant the area in which the patient is being examined, or treated. This is ordinarily the area surrounding the chair 15, bed 16, or table on which the patient 14 sits or lies while he or she is in the examination/treatment room 13. The ceiling newsletter 10 is within view of the patient 14 as he or she sits or lies on the chair 15, bed 16, or other type of furniture when the ceiling newsletter 10 is removably attached to the ceiling 12 of (over) the treatment area 11, as shown in FIGS. 1 through 3.

The brightly colored ceiling newsletter 10 is preferably brightly colored and appealing to attract the patient's attention. The ceiling newsletter is between one and four pages, preferably one or two pages. It has been found herein that each sheet of the ceiling newsletter issue should preferably be poster-sized, so as not to discourage the patient from reading it. Each newsletter page is most preferably between about two and three feet in width and between about two and three feet in length. Although a substantially square page (as viewed from the front or rear of the page) is preferred, a page can be rectangular (e.g., two feet in width and four feet in length). The ceiling newsletter 10 is preferably printed on sheets of paper, preferably between about 50 pound and 100 pound weight paper so it has integrity and is easy to mount on the ceiling, yet does not weigh so much that it falls off the ceiling. Each sheet, or page, of the ceiling newsletter has a front, printed side, or surface, that the patient views and an opposite back side that faces the ceiling.

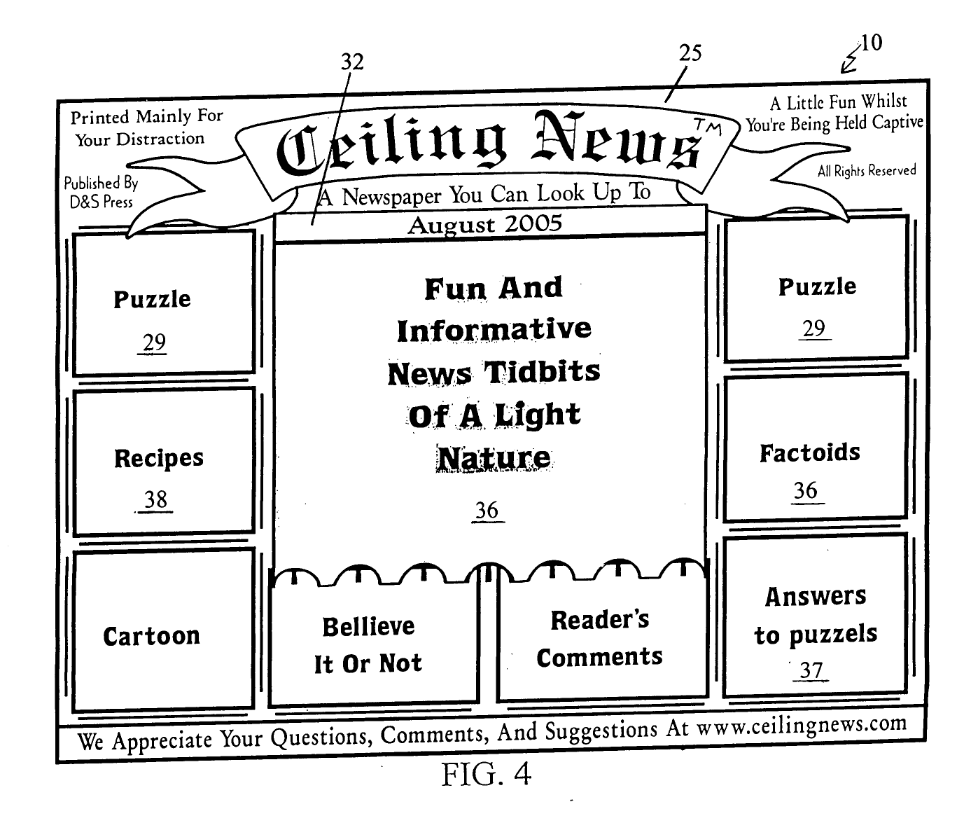

An illustrative example of a layout of a one page ceiling newsletter 10 is provided in FIG. 4. The ceiling newsletter 10, which is preferably issued on a monthly basis, is preferably sent to the consumer (dentist, dental hygienist, blood donation center, etc.) on a yearly subscription basis. The monthly issue of the newsletter 10 is preferably rolled into a cylindrical shape and mailed to the customer in a mailing tube. Attachment mechanism members 17, preferably at least two hook and loop strips, are packed in the mailing tube along with the issue of the ceiling newsletter 10.

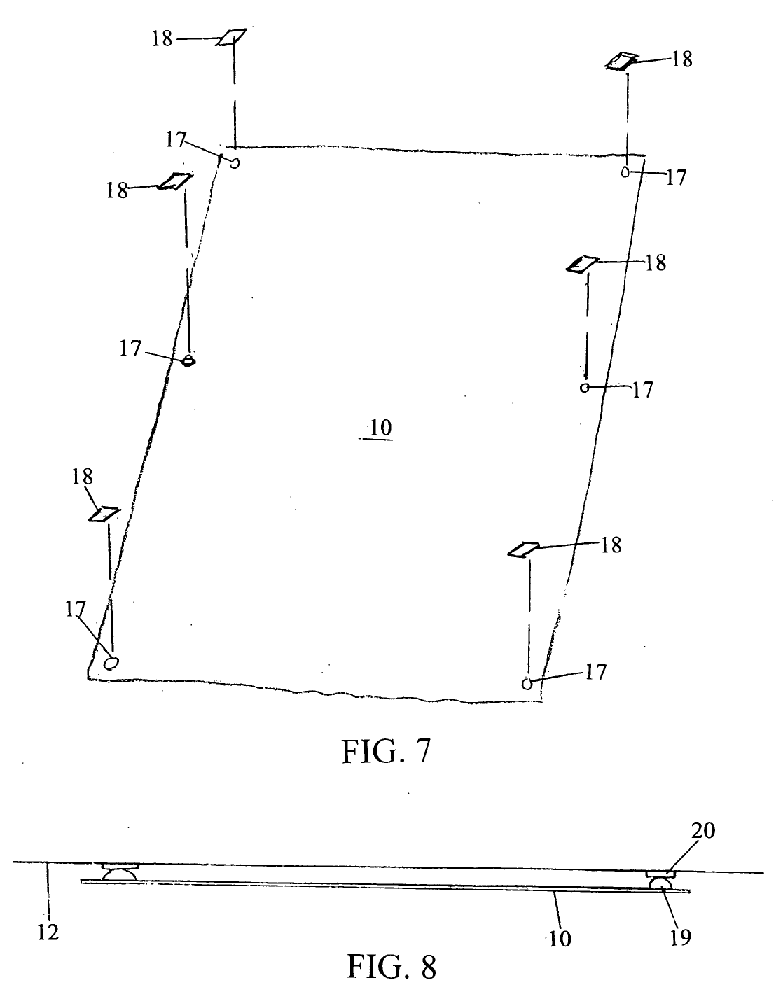

Directions are included with the first issue of the newsletter, along with a set of corresponding hook and loop strips 18. The directions specify the distance apart and arrangement of the corresponding hook and loop strips 18 on the ceiling 12. The backs of the corresponding, ceiling hook and loop strips 18 are applied to the ceiling 12 using an effective glue or by any other suitable means. The ceiling 12 need not be smooth (e.g., dry wall) for the present invention. Then, as each issue arrives, the consumer removes the ceiling newsletter 10 from the mailing tube, unrolls it, and attaches a hook and loop strip 17 to each corner of each page of the ceiling newsletter 10 (see FIG. 7). The hook and loop strip 17 preferably has a peel-off backing, with a suitable adhesive on the back of the hook and loop strip, so that each hook and loop strip is easy to apply to the back of the newsletter page.

The customer, or his or her assistant, then stands on a sturdy stool or the like and presses the hook and loop strips 17 on the back of each page of the ceiling newsletter 10 to the corresponding hook and loop strips 18 on the ceiling 12 of the treatment room 13, so that a patient 14 sitting in the treatment chair or lying on a bed or cot can view it (see FIGS. 1 through 3). A third set of hook and loop strips 17 may be attached to the back of the ceiling newsletter along its edge midway between the corners, as shown in FIG. 7. Once the ceiling newsletter 10 is mounted on the ceiling 12, it is generally parallel to the ceiling, as seen in FIG. 8. The ceiling newsletter 10 is easy to remove by pulling it down and replacing it with the next issue when it arrives.

Any other suitable attachment mechanism 17 may be employed in place of hook and loop strips. For example, at least two sets of inexpensive, mutually attracting magnets 19, 20 may be applied to the back of the newsletter 10 and the ceiling 12, respectively, in a corresponding pattern using glue or any other suitable means. The customer, or his or her helper, then stands on a sturdy stool or the like and places the magnets 19 on the back of each page of the ceiling newsletter 10 in proximity to the corresponding magnets 20 on the ceiling 12 of the treatment room 13 so that the magnets attach to one another. The same magnet pattern may be employed as is shown in FIG. 7, if desired. Once the ceiling newsletter 10 is mounted on the ceiling 12, it is generally parallel to the ceiling, as seen in FIG. 8. The ceiling newsletter 10 is easy to remove by pulling it down and replacing it with the next issue when it arrives. Since the ceiling newsletter 10 is lightweight, the magnets are thin and need not be powerful. Any suitable magnetized member may be employed. The magnetized portion may occupy a relatively small or large space on the back side of the newsletter page.

Alternatively, the ceiling newsletter 10 may arrive in a frame (not shown) with a number of hook and loop strips 17 or small magnets 19 already in place on the back of the frame. Once the customer applies the initial corresponding attachment mechanism 18 (e.g., corresponding hook and loop strips 18 or small magnets 20) to the ceiling 12, for each issue, the customer need only stand on a stool or the like and push the frame against the corresponding, ceiling attachment mechanism on the ceiling 12.

The ceiling newsletter 10 includes various copy and graphic elements, with color used throughout to highlight these elements and to add to the pleasing appearance of the newsletter. Sections commonly found in each month's issue of the newsletter include: jokes and riddles, puzzles, games, stories and facts pertaining to that particular month (for example, holiday features), trivia and memorabilia facts, translated phrases from a foreign language, travel tips, national charity drive announcements, easy recipes, helpful household hints, true or false questions or other questions with answers supplied, commonly used phrase origins (for example, “Boxer Day”), announcements of upcoming events, and/or an empty box (i.e., box is devoid of copy) for the customer to fill in with local announcements or the like (for example, “We are closed on Memorial Day”). In the latter case, the newsletter sheet is made of a material that can be marked on with a conventional writing instrument, such as paper.

The print on the ceiling newsletter 10 is larger than the print of a conventional newspaper. The font size for the copy in the ceiling newsletter is preferably between about 0.3 inch and one inch, so that the text can easily be read by a patient lying on the treatment table or sitting in the chair below the newsletter, assuming a patient with approximately 20/20 vision and a conventional ceiling height of about eight feet. Of course, a larger print size can be used for a higher (e.g., twelve foot) ceiling. The font size in the masthead 25 is preferably larger than the font size in the text (copy). The ceiling newsletter 10 preferably dos not contain a lot of text (copy), since it is intended to promote relaxation (using humor and light reading) rather than to convey complex news stories.

A preferred method according to the present invention includes the steps of: (a) providing at least one page of a periodically issued ceiling newsletter 10; (b) mounting at least two members 18 of an attachment mechanism on a ceiling 12 of the treatment area 11; (c) mounting at least two corresponding members 17 of the attachment mechanism on a back side of each issue of the at least one page ceiling newsletter 10; and (d) removably mounting the at least one page of the ceiling newsletter 10 on the ceiling 12 of the treatment area 11, with each of the at least one attachment mechanism members 17 corresponding to one of the corresponding attachment mechanism members 18 mounted on the ceiling 12. The method preferably further includes, between step (c) and step (d), the step of: removing a previous issue of the ceiling newsletter 10 from the ceiling 12 of the treatment area 11; wherein the current issue of the ceiling newsletter 10 is attachable to the ceiling attachment mechanism members 18 of step (b). Although the ceiling attachment mechanism members 17, 18 are hook and loop strips 17, 18 as described herein (see FIG. 7), they may alternatively be corresponding, mutually attracting magnets 19, 20 (see FIG. 8).

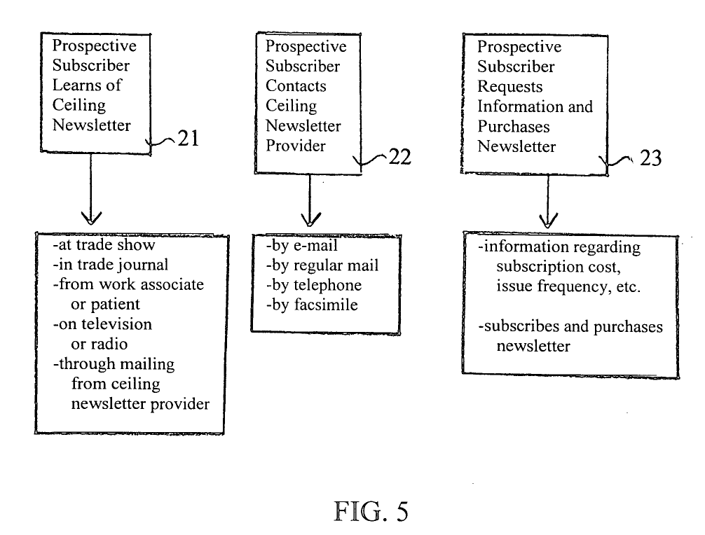

Referring to FIG. 5, a method 20 for reducing patient stress according to the present invention comprises the initial steps of:
A) Learning of the ceiling newsletter 10: at a trade show, from a work associate or another patient, from advertising on television, radio, magazine or trade journal, or other mass media, or a mailing from the ceiling newsletter provider, as shown in Block 21;
B) Contacting the ceiling newsletter provider, preferably by e-mail, by United States Postal Service mail, by telephone, or by facsimile, as shown in Block 22;
C) Requesting information regarding subscription cost, issue frequency, or other information, and subscribing and purchasing, preferably by credit card, check, money order, or other means, as shown in Block 23.

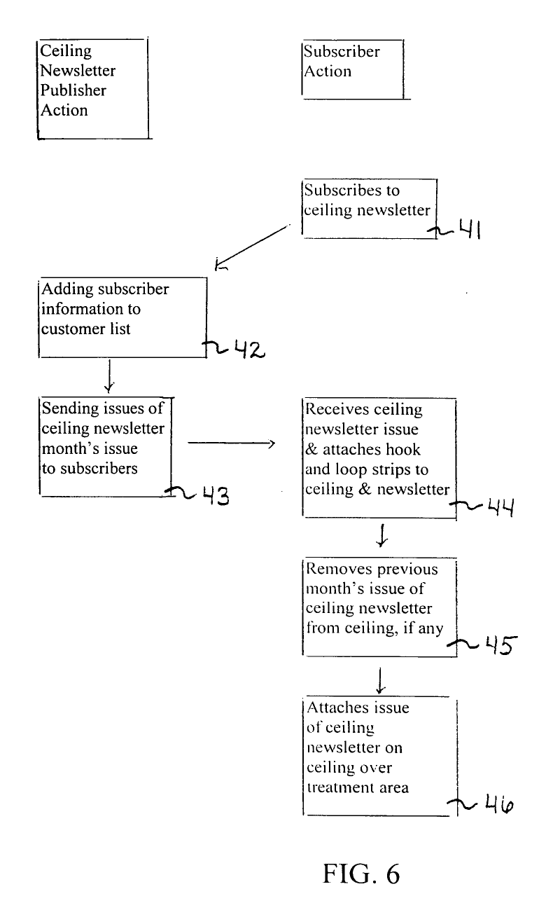

Referring to FIG. 6, a method according to the present invention for reducing stress on a patient about to undergo or undergoing a stressful treatment (including a medical or dental examination), comprising the steps of:
a) Subscribing to the ceiling newsletter, as shown in Block 41, preferably via the website or mail;
b) Transcribing subscriber information from a subscription order form to a customer list, as shown in Block 42;
c) Mailing the issue of the ceiling newsletter 10 to each subscriber periodically, as shown in Block 43, preferably on or about the 15th day of every month (each mailing is the next month's issue), most preferably in a mailing tube; and
d) Receiving the issue of the next month's ceiling newsletter 10, preferably by the first of the month, unpacking it, attaching the attachment mechanism members 17, 18 to the ceiling 12 of the treatment area and to the back of the ceiling newsletter 10, as shown in Block 44, and attaching this current (new) issue of the ceiling newsletter 10 to the ceiling 12 of the treatment area 11, as shown in Block 46. Step d) preferably further comprises: removing the previous month's issue of the ceiling newsletter 10, if any, from the ceiling 12 of the treatment area 11, as shown in Block 45, before attaching the current issue to the ceiling. New ceiling newsletters 10 are sent to customers on a monthly basis. Patients of ceiling newsletter customers are unlikely to be bored, and are more likely to relax and less likely to worry as they await or undergo treatment.

Of these steps, Step c) preferably further comprises: including a ceiling attachment mechanism for attaching the newsletter 10 to the ceiling 12, preferably at least four hook and loop strips 17, along with the issue of the ceiling newsletter 10. Step c) preferably further comprises: including at least one set of “miniatures” 10b along with the larger ceiling newsletter for the occasional patient who wishes to take a copy home with them. By “miniatures” 10b is meant a miniature duplicate, most preferably an 8½ inch wide by 11 inch long sheet or sheets, of the ceiling newsletter 10. Once the patient sees the ceiling newsletter 10 in the examination room and expresses an interest in taking it home, the customer offers the patient the miniature 10b, or a copy of the miniature, to take home with the patient. This usually pleases the patient and helps to promote good business relations.

In Step d), the attachment mechanism members are more preferably hook and loop strips 17 affixed to the corners of the back side of the ceiling newsletter 10. These hook and loop strips 17 on the ceiling newsletter 10 are attached to corresponding hook and loop strips 18 previously attached to the ceiling 12 of the treatment area 11. The hook and loop strips 17, 18 are depicted in FIG. 7. FIG. 8 shows the ceiling newsletter 10 once it has been attached to a ceiling.

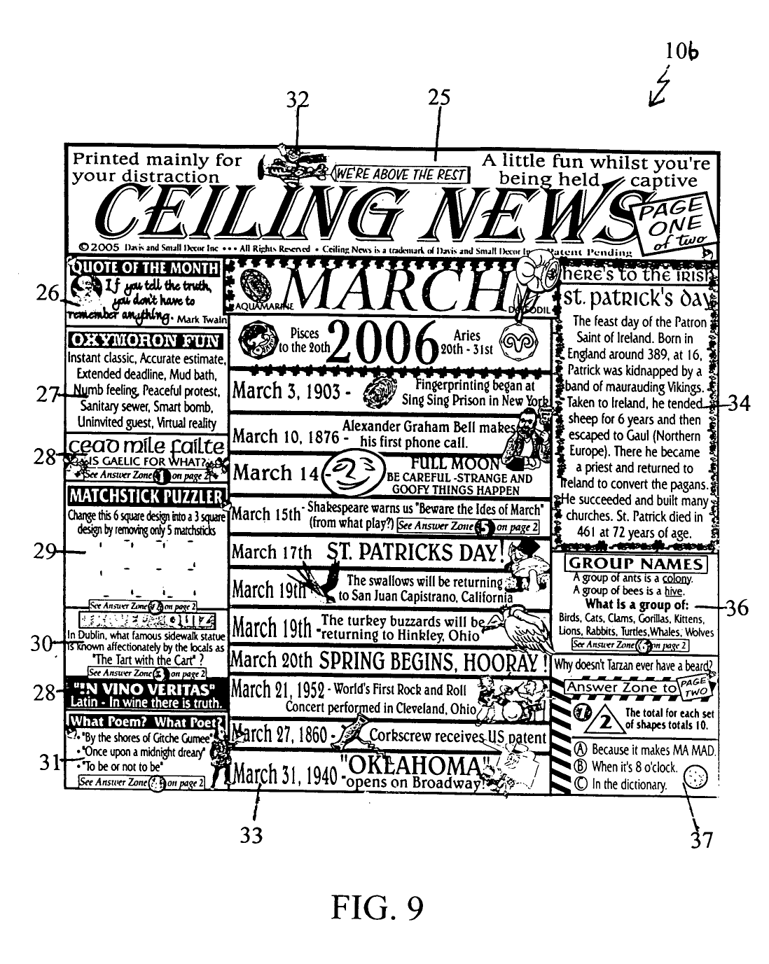

A set of miniatures 10b is shown in FIGS. 9 (first page) and 10 (second page). These miniatures mirror the full size ceiling newsletter 10. Each page of the miniature 10b is preferably the size of a conventional sheet of paper (8½ by 11 inches).

The monthly issue of the ceiling newsletter is preferably two pages long. The two pages are preferably attached to the ceiling in a side by side arrangement (see FIGS. 1 and 2) or an end to end arrangement (see FIG. 3), although the two pages can be attached to the ceiling diagonal to one another.

Thus, the present newsletter system for easing stress on a patient in a high stress treatment area, includes:
(a) at least one page of a periodically issued ceiling newsletter 10;
(b) at least two sets of hook and loop strips 17 (preferably six sets) attachable to the back of the page of the ceiling newsletter 10;
(c) at least two sets of corresponding hook and loop strips 18 attachable to a ceiling 12 over the treatment area 11; and
(d) at least one miniature 10b of the ceiling newsletter 10, for the customer to give to an inquiring customer. The ceiling newsletter 10 is removably mountable on the ceiling 12 over the treatment area 11. The ceiling newsletter 10 is preferably issued on a monthly basis.

As shown in FIG. 9, a preferred embodiment of the ceiling newsletter 10 includes the following: a) a masthead 25 across the top of the ceiling newsletter 10 providing the name of the newsletter, which is preferably “Ceiling News”; b) a different, brief “Quote of the Month” 26 each month; c) at least one oxymoron or palindrome fun 27 with a different oxymoron or palindrome each month; d) at least one translated phrase 28 from a foreign language, preferably Gaelic; e) at least one brief puzzle 29; f) at least one short quiz 30 or game; g) at least one short poem 31; h) the particular month 32 and year in which the ceiling newsletter is printed, preferably with an outline of a flower or symbol representing that month; i) a calendar of events 33 in that month; j) a short story 34 pertaining to that month; k) at least one piece of trivia or fun fact 36; and/or l) an “Answer Zone” 37 containing the answers to the questions on the opposite page of the ceiling newsletter. The Answer Zone is preferably on the opposite page from the questions so the patient will not be tempted to “cheat”. The masthead 25 preferably also includes the slogans “We're above the rest” and/or “A little fun whilst you're being held captive”. The masthead 25 and slogans are preferably used on every issue of the ceiling newsletter 10, and every page, to build customer recognition.

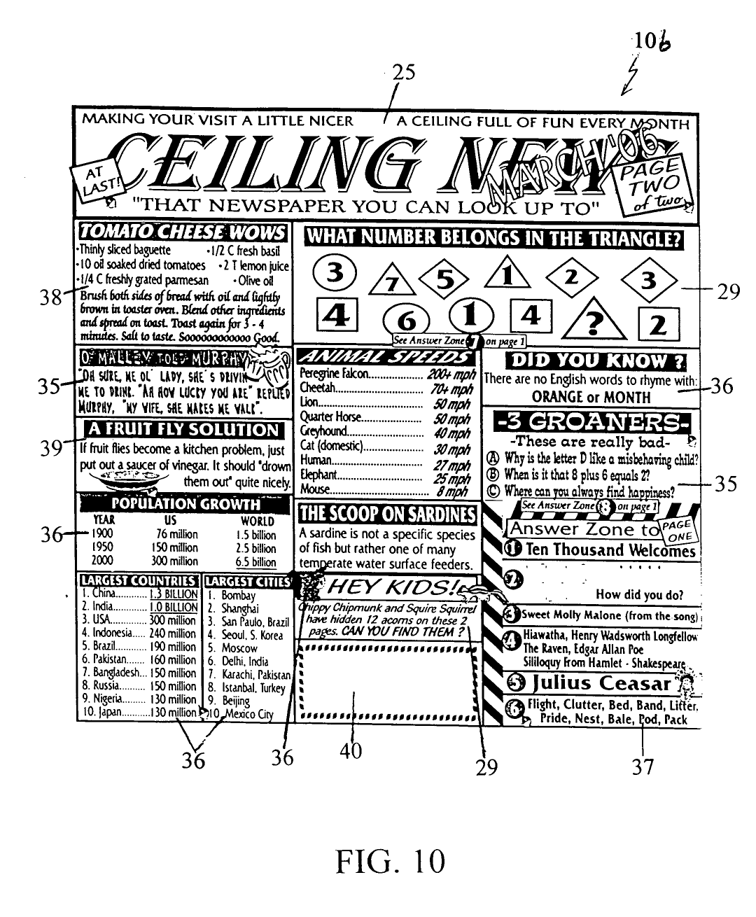

Continuing with FIG. 10, the ceiling newsletter 10 preferably further includes the following, which are preferably in individual boxes to facilitate reading: m) at least one joke 35 or riddle; n) at least one short, easy recipe 38; o) household hints 39; and/or p) an empty box 40 for the customer to fill in with local announcements or the like (for example, “We are closed on Memorial Day”). These are preferably bordered by individual boxes to facilitate reading. The ceiling newsletter may also include travel tips, national charity drive announcements, cartoons, reader's comments, an email address and solicitation of reader comments or questions, and/or true or false questions or other questions.

From the foregoing it can be realized that the described method of the present invention may be easily and conveniently utilized to display the ceiling newsletter of the present invention on a ceiling over a treatment area or the like. It is to be understood that any dimensions given herein are illustrative, and are not meant to be limiting.

While preferred embodiments of the invention have been described using specific terms, this description is for illustrative purposes only. It will be apparent to those of ordinary skill in the art that various modifications, substitutions, omissions, and changes may be made without departing from the spirit or scope of the invention, and that such are intended to be within the scope of the present invention as defined by the following claims. It is intended that the doctrine of equivalents be relied upon to determine the fair scope of these claims in connection with any other person's product which fall outside the literal wording of these claims, but which in reality do not materially depart from this invention. Without further analysis, the foregoing will so fully reveal the gist of the present invention that others can, by applying current knowledge, readily adapt it for various applications without omitting features that, from the standpoint of prior art, fairly constitute essential characteristics of the generic or specific aspects of this invention.

What is claimed is:

1. A method for easing stress on a patient in a stressful treatment area, the method comprising the steps of:
(a) providing at least one page of a periodically issued ceiling newsletter;
(b) mounting at least two members of an attachment mechanism on a ceiling of the treatment area;
(c) mounting at least two corresponding members of the attachment mechanism on a back side of each issue of the at least one page ceiling newsletter; and
(d) removably mounting the at least one page of the ceiling newsletter on the ceiling of the treatment area, with each of the at least one attachment mechanism members corresponding to one of the corresponding attachment mechanism members mounted on the ceiling.

2. The method according to claim 1, further comprising between step (c) and step (d) the step of: removing a previous issue of the ceiling newsletter from the ceiling of the treatment area; wherein the current issue of the ceiling newsletter is attachable to the ceiling attachment mechanism members of step (b).

3. The method according to claim 1, wherein the ceiling attachment mechanism members are hook and loop strips.

4. The method according to claim 1, wherein the ceiling attachment mechanism members are corresponding, mutually attracting magnets.

5. A method for reducing stress on a patient about to undergo or undergoing a stressful treatment, the method comprising the steps of:
a) Subscribing to a ceiling newsletter;
b) Transcribing subscriber information from the new subscriber to a customer list;
c) Mailing an issue of the ceiling newsletter and corresponding attachment mechanism members to each subscriber periodically; and
d) Receiving the issue of the ceiling newsletter, affixing corresponding attachment mechanism members to the ceiling of a treatment area and to the back of each page of the ceiling newsletter, and removably attaching the issue of the ceiling newsletter to the treatment area ceiling.

6. The method according to claim 5, preceded by the initial steps of:
A) Learning of the ceiling newsletter: at a trade show; from a work associate or another patient; from advertising on television, radio, magazine or trade journal, or other mass media, or a mailing from the ceiling newsletter provider;
B) Contacting the ceiling newsletter provider; and
C) Requesting information regarding subscription cost issue frequency, or other information, and purchasing a ceiling newsletter subscription.

7. The method according to claim 5, further comprising the step in Step d) of: removing a previous issue of the ceiling newsletter from the ceiling of the treatment area before attaching the current issue of the ceiling newsletter to the ceiling.

8. The method according to claim 5, wherein step c) comprises rolling the ceiling newsletter into a cylindrical shape and mailing it and the attachment mechanism members to the customer in a mailing tube.

9. The method according to claim 5, wherein Step c) preferably further comprises: including a ceiling attachment mechanism with the issue of the ceiling newsletter.

10. The method according to claim 5, wherein Step c) preferably further comprises: including at least one set of ceiling newsletter miniatures mirroring the larger ceiling newsletter along with the corresponding ceiling newsletter.

11. The method according to claim 7, wherein the ceiling attachment mechanism is at least two sets of hook and loop strips.

12. The method according to claim 5, wherein the ceiling attachment mechanism is at least two sets of corresponding, mutually attracting magnets.

13. The method according to claim 5, wherein the corresponding attachment mechanism members are mounted to the ceiling by an adhesive.

14. The method according to claim 11, wherein Step d) preferably further comprises affixing the hook and loop strips to the corners on back of each page of the ceiling newsletter, and attaching the hook and loop strips on the ceiling newsletter to corresponding hook and loop strips mounted on the ceiling over the treatment area.

15. A newsletter system for easing stress on a patient in a high stress treatment area, the system comprising:
(a) at least one page of a periodically issued ceiling newsletter;
(b) at least two sets of ceiling attachment mechanism members attachable to a back of the at least one page of the ceiling newsletter;
(c) at least two sets of corresponding ceiling attachment mechanism members attachable to a ceiling over the treatment area; and
(d) at least one miniature of the ceiling newsletter;
wherein the at least one page of the ceiling newsletter is removably mountable on the ceiling over the treatment area.

16. The system according to claim 15, wherein the ceiling attachment mechanism is at least two sets of hook and loop strips.

17. The system according to claim 15, wherein the ceiling attachment mechanism is at least two sets of mutually attracting magnets.
